(19) United States
(12) Patent Application Publication
(10) Pub. No.: US 2003/0224388 A1
(43) Pub. Date: Dec. 4, 2003

(54) RIP2: a mediator of signaling in the innate and adaptive immune systems

(76) Inventors: Richard A. Flavell, Guilford, CT (US); Ruslan M. Medzhitov, Branford, CT (US); Koichi Kobayashi, Branford, CT (US)

Correspondence Address: ROPES & GRAY LLP, ONE INTERNATIONAL PLACE, BOSTON, MA, 02110-2624, US

(21) Appl. No.: 10/339,636

(22) Filed: Jan. 9, 2003

Related U.S. Application Data
(60) Provisional application No. 60/348,172, filed on Jan. 9, 2002.

(51) Int. Cl. C12Q 1/68; G01N 33/53; G01N 33/567

(52) U.S. Cl. 435/6; 435/7.1; 435/7.2

(57) ABSTRACT

This invention provides a method of identifying a compound that modulates an innate immune response and an adaptive immune response comprising contacting cells expressing RIP2 with a candidate compound, and determining whether the candidate compound modulates RIP2 activity in the cells, wherein modulation of RIP2 activity in the cells by the candidate compound indicates that the candidate compound modulates the innate immune response and adaptive immune response.

### RELATED APPLICATIONS

This application claims the benefit of the filing date of U.S. Provisional Application No. 60/348,172, filed Jan. 9, 2002 and entitled “RIP2/RICK/CARDIAK mediates signaling for receptors of both the innate and adaptive immune systems,” by Richard A. Flavell, Koichi Kobayashi and Ruslan Medzhitov. The entire teachings of the referenced provisional application are incorporated herein by reference.

Throughout this application, various publications are referenced, either by Arabic numerals or directly in the text. Full citations for those publications referenced by Arabic numerals may be found at the end of the specification immediately proceeding the claims. The disclosure of all referenced publications is hereby incorporated by reference into this application to describe more fully the art to which this invention pertains.

### FUNDING

Work described herein was supported by National Institutes of Health grant number PO1 AI 36529 The United States Government has rights in the invention.

### BACKGROUND OF THE INVENTION

The immune system consists of two evolutionarily different but closely related aims: innate immunity and adaptive immunity. Each has characteristic receptors: toll like receptors (TLRs) and Nod protein family members and antigen-specific receptors, respectively. A better understanding of immune system regulation would provide opportunities to develop approaches to modulating immune response.

### SUMMARY OF THE INVENTION

Applicants have shown that the CARD containing serine/threonine kinase, RIP2 (also known as RICK, CARDIAK or CCK) transduces signals from receptors of both of these immune systems. Cytokine production in RIP2-deficient cells was significantly reduced upon stimulation of TLRs with LPS, peptidoglycan, and double-stranded RNA but not with bacterial DNA, indicating that RIP2 is downstream of some TLRs (e. g. TLR4, the receptor for LPS) but not others (e. g. TLR9, the receptor for bacterial DNA). RIP2-deficient cells were hyporesponsive to IL-1β and IL-18 stimulation, suggesting that RIP2 is involved in signaling through the evolutionarily conserved TLR/IL-1 receptor family. RIP2-deficient cells were deficient for signaling through Nod proteins, which have also been implicated in the innate immune response. Finally, T cells from RIP2 deficient mice showed severely reduced proliferation and IL-2 production upon T cell receptor (TCR) engagement, which was accompanied by impaired activation of NF-κB. Th1 differentiation was also perturbed in RIP2 deficient T cells, indicating that RIP2 is required for TCR signaling and T cell differentiation. Together these results show that RIP2 is a unique kinase that acts as a signal transducer and integrator of signals for both the innate and adaptive immune systems. Because RIP2 is able to integrate signals from both the innate and the adaptive immune systems, RIP2 is a unique target for modulating or altering the immune response.

The preferred methods and materials are described below in examples which are meant to illustrate, not limit, the invention. Skilled artisans will recognize methods and materials that are similar or equivalent to those described herein, and that can be used in the practice or testing of the present invention.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, the preferred methods and materials are described herein.

The details of one or more embodiments of the invention are set forth in the accompanying drawings and the description below. Other features, objects, and advantages of the invention will be apparent from the description and drawings, and from the claims.

The practice of the present invention will employ, unless otherwise indicated, conventional techniques of cell biology, cell culture, molecular biology, transgenic biology, microbiology, recombinant DNA, and immunology, which are within the skill of the art. Such techniques are explained fully in the literature. See, for example, Molecular Cloning A Laboratory Manual, 2nd Ed., ed. by Sambrook, Fritsch and Maniatis (Cold Spring Harbor Laboratory Press: 1989); DNA Cloning, Volumes I and II (D. N. Glover ed., 1985); Oligonucleotide Synthesis (M. J. Gait ed., 1984); Mullis et al. U.S. Pat. No: 4,683,195; Nucleic Acid Hybridization (B. D. Hames & S. J. Higgins eds. 1984); Transcription And Translation (B. D. Hames & S. J. Higgins eds. 1984);Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); the treatise, Methods In Enzymology (Academic Press, Inc., N.Y.); Gene Transfer Vectors For Mammalian Cells (J. H. Miller and M. P. Calos eds., 1987, Cold Spring Harbor Laboratory); Methods In Enzymology, Vols. 154 and 155 (Wu et al. eds.), Immunochemical Methods In Cell And Molecular Biology (Mayer and Walker, eds., Academic Press, London, 1987); Handbook Of Experimental Immunology, Volumes I-IV (D. M. Weir and C. C. Blackwell, eds., 1986); Manipulating the Mouse Embryo, (Cold Spring Harbor Laboratory Press, Cold Spring Harbor, N.Y., 1986).

### BRIEF DESCRIPTION OF THE DRAWINGS

FIGS. 1a-1d: Expression of RIP2 in macrophages and targeted disruption of the mouse Rip2 gene. FIGS. 1a and 1b: Northern blot analysis and Western blot analysis for RIP2 expression in macrophages. Macrophages were stimulated with 10 ng/ml of LPS for the indicated periods, and total RNA and protein samples were obtained. Northern blot and Western blot analysis were performed using RIP2 and hypoxanthine guanine phosphoribosyl transferase) (“HPRT”) specific probes, and anti-RIP2 antibody respectively. FIG. 1c: A schematic diagram of the Rip2 locus, the targeting vector and the targeted allele. Filled boxes denotes the coding exons. Restriction enzyme sites are indicated (S, SacI; EV, EcoRV; X, XbaI; A, Apal). TK=typmidine kinase. FIG. 1d: Southern blot analysis of genomic DNA identifies mice corresponding to all three expected genotypes. SacI digested DNA was probed as indicated. The upper band (9.5 kb) corresponds to the wild-type allele, and the lower band (7.3 kb) to the mutant allele. FIG. 1e: Western blot analysis of thymocytes showing the absence of RIP2 protein in homozygous mice.

FIGS. 2a-2d: Impaired TLR responses in RIP2-deficient cells. FIG. 2a: Bone marrow derived macrophages were stimulated with LPS (10 ng/ml), Lipoteichoic acid (LTA, 10 μg/ml), peptidoglycan (PGN, 10 μg/ml), CpG oligo DNA (CpG, 10 μM) and medium alone (MED) for 6 hours and the concentration of secreted IL-6, TNFα, and IP10 was measured by ELISA. The figures are the representatives of six independent experiments for IL-6 and three independent experiments for TNFα and IP10. FIG. 2b: Macrophages were infected with Listeria monocyotogenes for 30 min, washed with DPBS twice to eliminate unattached bacteria and gentamicin was added to prevent recurrent infection. Cells were cultured for the indicated period, or cultured for 6 hours in the presence of the indicated concentrations of cytochalasin D. The concentration of IL-6 in the supernatant was measured. The figures are the representative of the two independent experiments. Both showed similar results. FIG. 2c: Fibroblasts from wild-type or RIP2^{-/-} embryos were stimulated with poly(IC) or LPS with indicated dose for 24 hours, or stimulated with 100 μg/ml of poly(IC) or 10 ng/ml of LPS for indicated periods. IL-6 level was measured as in a. The figures are the representative of the two independent experiments. Both showed similar results. FIG. 2d: Survival curve of wild-type and RIP2^{-/-} mice for endotoxin shock. 16.7 mg/kg body weight LPS was injected intraperitoneally into wild-type and RIP2-/- mice. Mice were observed every 12 hours for 5 days. There was no incremental death after 80 hours. P values were determined by the Mantel-Cox test.

FIGS. 3a-3c: RIP2 is associated with TLR signaling complexes. FIG. 3a: Coimmunoprecipitation of TLR2/4 and RIP2. Myc-tagged TLR2ΔLRR or TLR4ΔLRR expression vectors were cotransfected with FLAG-tagged RIP2 into 293T cells. 24 hours later, cell lysates were prepared and immunoprecipitated with anti-Flag antibody. Associated molecules were detected with anti-Myc antibody. FIG. 3b: Coimmunoprecipitation of MyD88 and RIP2. Myc-tagged TLR4ΔLRR or RIP2 expression vectors were cotransfected with FLAG-tagged MyD88 into 293T cells. 24 hours later, cell lysates were prepared and immunoprecipitated with anti-Flag antibody. Associated molecules were detected with anti-Myc antibody. FIG. 3c: RIP2 is upstream of multiple signaling pathways including NF-κB, JNK, p38 and ERK. Bone marrow macrophages of wild-type and RIP2-deficient mice were stimulated with LPS (10 ng/ml) for indicated periods. Cell lysates were prepared and blotted with anti-phospho-IκBα, anti-IκBα, anti-phospho-JNK, anti-JNK, anti-phospho-p38, anti-p38 and anti-phospho-ERK1/2 and anti-ERK1/2 antibodies. Data were scanned and quantified by an imageanalyzer. Ratios of phosphorylated proteins and unphosphorylated proteins are shown. The data is mean of three independent experiments. The ratios of p-JNK/JNK represent those of the upper band. The ratios of the lower band are 0.00, 0.04, 0.54, 0.00 in wild-type and 0.00, 0.07, 0.27, 0.00 in RIP2^{-/-} cells.

FIG. 4: RIP2 is essential for activation of NF-κB by Nod1 and Nod2. Embryonic fibroblasts from wild-type and RIP2-deficient mice were co-transfected with pcDNA3, pcDNA3-IKKβ, pcDNA3-Nod1dLRR, pcDNA3-Nod2, pcDNA3-RIP2 or pcDNA3-DN-MyD88 and pEF-BOS-β-gal and pBVI-luc reporter plasmids. For LPS stimulation, fibroblasts were cotransfected with pEF-BOS-β-gal and pBVI-luc reporter plasmids and stimulated with LPS (10 μg/ml) for 6 hours.

FIGS. 5a-5c: RIP2 is required for optimal IL-1/IL-18 receptor signaling. FIG. 5a: RIP2 is required for proliferation of thymocytes stimulated with IL-1β. Thymocytes from wild-type and RIP2^{-/-} mice were stimulated with IL-2 (2 ng/ml) or Con A (0.625 μg/ml) alone or together with IL-1β (10 ng/ml) and cultured for indicated periods. Cells were pulsed with [³H] thymidine 8 hours before harvest. The experiments were performed twice in triplicate. Both showed similar results. FIG. 5b: IL-6 production by embryonic fibroblasts stimulated with cytokines. Fibroblasts from wild-type and RIP2^{-/-} embryo were stimulated with IL-1β (10 ng/ml) or TNFα (10 ng/ml) and cultured for indicated periods. The concentration of IL-6 in the supernatant was measured. The experiments were performed twice in triplicate. Both showed similar results. FIG. 5c: Production of IFNγ by NK cells upon stimulation with IL-18 and IL-12. Splenocytes from wild-type or RIP2^{-/-} mice were stimulated with IL-18 (10 ng/ml) or IL-12 (10 ng/ml) alone for 48 hours or with combination of both IL-18 (10 ng/ml) and IL-12 (1 ng/ml) for 24 hours. The concentration of IFNγ in the supernatant was measured. The figures are representative of three independent experiments. FIG. 5d: Production of IFNγ by Th1 cells upon IL-18 and IL-12 stimulation. Purified CD4⁺ T cells from wild-type and RIP₂^{-/-} mice were stimulated with plate-bound anti-CD3 (10 mg/ml) and cultured in Th1 condition for 4 days. Cells were washed, counted and stimulated with either IL-18 (10 ng/ml) or IL-12 (10 ng/ml) alone, or combination of IL-18 and IL-12 for 24 hours. The concentration of IFNγ in the supernatant was measured. The figures are representative of three independent experiments. FIG. 5e: Th1/Th2 differentiation of CD4⁺ T cells in vitro. CD4⁺ T cells from wild-type or RIP2^{-/-} mice were stimulated with 10 μg/ml of plate-bound anti-CD3 and cultured for 4 days under Th1 conditions (in the presence of 3.5 ng/ml of IL-12 and 2 μg/ml of anti-IL-4 antibody) or in Th2 condition (Th2; in the presence of 1000 U/ml of IL-4 and 1 μg/ml of anti-IFNγ antibody). After washing, cells were counted and restimulated with 10 μg/ml of anti-CD3 and cultured for 24 hours. The concentration of IFNγ and IL-4 in the supernatant was measured. The figures are representative of three independent experiments.

FIGS. 6a-6d: RIP2 is required for NF-κB activation and T cell proliferation upon TCR stimulation. FIG. 6a: Proliferation assay of CD4⁺ T cells. CD4⁺ T cells from wild-type and RIP2^{-/-} mice were stimulated with Concanavalin A (Con A, 2.5 μg/ml), PMA (40 ng/ml)+Ionomycin (0.5 μM) or anti-CD3 with indicated dose for 96 hours, or with 10 μg/ml of anti-CD3 for indicated periods in the presence of irradiated T cell depleted splenocytes. Cells were pulsed with [³H] thymidine 8 hours before harvest. The figures are representative of three independent experiments. FIG. 6b: IL-2 production by CD4⁺ T cells upon anti-CD3 stimulation. CD4⁺ T cells were stimulated with plate-coated anti-CD3 with the indicated dose either in the absence or presence of anti-CD28 (2 μg/ml), or with Con A (2.5 μg/ml). The figures are representative of three independent experiments. FIG. 6c: Phosphorylation and degradation of IκBα upon anti-CD3 stimulation. Splenic T cells from wild-type and RIP2-/- mice were stimulated with anti-CD3 (10 μg/ml) for indicated periods. Cell lysates were prepared and blotted with anti-phospho-IκBα and anti-IκBα. FIG. 6d: NF-κB activation upon anti-CD3 stimulation requires RIP2. Splenic T cells from wild-type and RIP2-/- mice were stimulated with anti-CD3 with indicated dose for 8 hours and nuclear lysates were prepared. NF-κB activation was analyzed by Gel Mobility Shift Assay using [³²P]dCTP-labeled, NF-κB binding site specific probe (5′-GAGTTGAGGGGACTTTCCCAGGC).

### DETAILED DESCRIPTION OF THE INVENTION

Unless defined otherwise, all technical and scientific terms have the same meaning as is commonly understood by one of skill in the art to which this invention belongs.

The articles “a,” “an” and “the” are used herein to refer to one or to more than one (i.e., to at least one) of the grammatical object of the article.

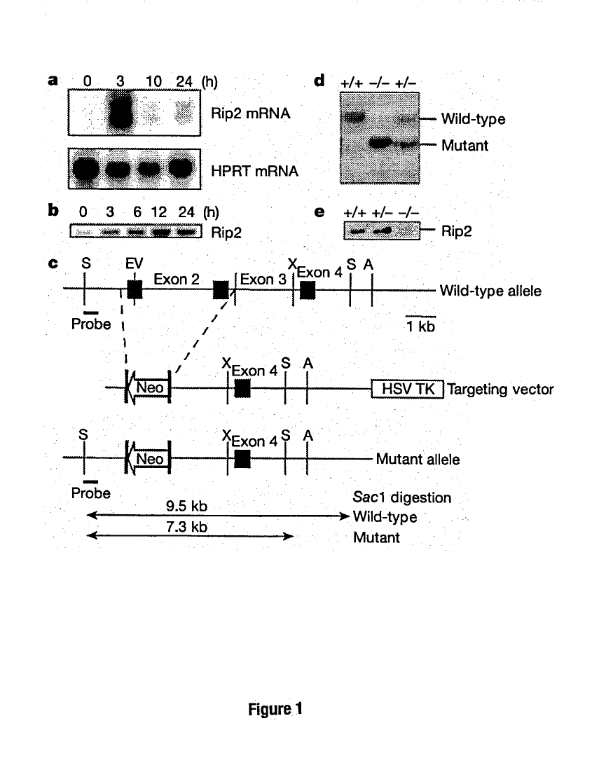

RIP2/RICK/CARDIAK/CCK ¹⁻⁴ is a serine/threonine kinase that carries a CARD at its C-terminus and shares sequence similarity with a serine/threonine kinase, RIP, essential for NF-κB activation via the TNF receptor ⁵. In vitro studies have shown that RIP2 can associate with a variety of other CARD-containing molecules via CARD-CARD interactions ¹⁻³. Moreover, overexpression of RIP2 causes activation of NF-κB and JNK ¹⁻⁴. NF-κB activation by RIP2 is inhibited by dominant negative TRAF6 ⁴, a signaling molecule downstream of TLRs. Expression of RIP2 was induced in macrophages upon stimulation with LPS (FIGS. 1a, b). These observations led Applicants to consider the possibility that RIP2 is involved in signaling in the innate immune system. To assess the physiological role of RIP2in the innate immune signaling, Applicants generated RIP2-deficient mice by homologous recombination of embryonic stem (ES) cells. A gene-targeting construct was generated to replace the two exons encoding murine RIP2 with a neomycin-resistance gene (neo) (FIG. 1c). Homologous recombination in ES cells was confirmed by Southern blot analysis (FIG. 1d), and the absence of RIP2 expression in homozygous animals was confirmed by Western blot (FIG. 1e). RIP2-deficient mice were born in the expected Mendelian ratio and showed no gross developmental abnormalities and no abnormal composition of lymphocytes as determined by flow cytometry.

This invention provides a method of identifying a compound that modulates an innate immune response and an adaptive immune response comprising contacting cells expressing RIP2 with a candidate compound, and determining whether the candidate compound modulates RIP2 activity in the cells, wherein modulation of RIP2 activity in the cells by the candidate compound indicates that the candidate compound modulates the innate immune response and adaptive immune response.

“A cell that does not express RIP2” as used herein refers to any cell that does not express RIP2. RIP2, as used herein, refers to the wild-type RIP2 having RIP2 activity. As used herein, cells that do not express RIP2 include cells that do not comprise a nucleic acid encoding RIP2, as well as cells comprising a nucleic acid encoding wild-type or mutant RIP2, but that do not have RIP2 activity. Cells that do not express RIP2 include RIP2-deficient cells and RIP2^{-/-} cells. Cells that do not express RIP2 can be obtained according to any method known to a person having ordinary skill in the art. In one embodiment cells that do not express RIP2 are obtained through homologous recombination. In one embodiment, a cell that does not express RIP2 can be obtained from a transgenic “knockout” animal that fails to express RIP2. Transgenic knockout animals can be made according to any method known to a person of skill in the art. See e.g., Silver, Mouse Genetics Concepts and Applications, Oxford University Press (1995); Kuida et al., Cell 94:325-337 (1998); Alexopoulou et al., Nature Medicine, 8(8): 872-884 (2002); Lu et al., Immunity, 14: 583-590 (2001).

“Knockout” animals refers to animals whose native or endogenous RIP2 allele or alleles have been disrupted by homologous recombination and which produce no functional RIP2 of their own. Knockout animals may be produced in accordance with techniques known in the art, particularly by means of in vivo homologous recombination, M. Capechi, Science 244, 1288-1292 (1989), in light of the known sequence for DNA encoding the RIP2. Sequences encoding mouse RIP2 include, but are not limited to, GenBank Accession Nos. AF461040 and AAL96436. Sequences encoding the human RIP2 include, but are not limited to, GenBank Accession Nos. AF078530 amd AAC27722.

The term “compound,” as used herein, can be any chemical or biological agent. Some examples of such test agents are synthetic chemicals, naturally occurring chemicals, proteins (e.g., polypeptides, antibodies), nucleotides (e.g., antisense oligonucleotides, interference RNA oligonucleotides), etc.

The term “modulator,” as used herein, refers to a compound that alters the function or activity of RIP2. The “modulator” can be an inhibitor, an activator, or an inducer a RIP2, or a combination thereof. An “inhibitor” is a compound that can inhibit or decrease the activity of RIP2. An “activator” is a compound that can increase the activity of RIP2. An “inducer” is a compound that can increase the expression of the RIP2, therefore increasing the activity of RIP2.

As used herein “contacting,” such as when used in the context of contacting a cell with a compound, refers to combining, mixing, or in any way bringing together a cell and a compound.

In one embodiment of this method, the contacting is conducted under conditions appropriate for entry of the candidate compound into the cells. In one embodiment of this method, the method further comprises the step of comparing the RIP2 activity in the presence of the candidate compound with the RIP2 activity of a standard known to be deficient in RIP2 activity, wherein RIP2 activity in the presence of the candidate compound which is comparable to RIP2 activity of the known standard indicates that the compound is a RIP2 inhibitor. The meaning of “comparable” as used herein may be explained with reference to the above embodiment. For example, the RIP2 activity in the presence of the compound is comparable to the RIP activity for the known standard of the magnitude between the RIP2 activity in the presence of the component and the RIP2 activity for the known standard is less than the difference in magnitude between the RIP2 activity in the presence and absence of the compound. In one embodiment of this method, the standard is the RIP2 activity determined in a cell which does not express RIP2. In one embodiment of this method, the modulator is an inhibitor of RIP2 activity, and inhibition of RIP2 activity in the cells by the candidate compound indicates that the candidate compound inhibits the innate immune response and adaptive immune response. In one embodiment of this method, the innate immune response is production of inflammatory cytokines and the adaptive immune response is production of antibodies.

This invention provides a method of identifying a compound that produces an anti-inflammatory effect and an immuno-inhibitory effect comprising contacting cells expressing RIP2 with a candidate compound and determining whether the candidate compound inhibits RIP2 activity in the cells, wherein inhibition of RIP2 activity in the cells by the candidate compund indicates that the candidate compound inhibits RIP2 activity, thereby identifying a compound that produces an anti-inflammatory effect and an immuno-inhibitory effect.

In one embodiment of this method, the contacting is conducted under conditions appropriate for entry of the candidate compounds into the cells. In one embodiment of this method, the method further comprises the step of comparing the RIP2 activity in the presence of the candidate compound with the RIP2 activity for a standard known to be deficient in RIP2 activity, wherein RIP2 activity in the presence of the candidate compound which is comparable to RIP2 activity for the known standard indicates that the compound is a RIP2 inhibitor. In one embodiment of this method, the standard is the RIP2 activity determined in a cell which does not express RIP2.

This invention provides a method of determining whether a compound is a RIP2 inhibitor comprising comparing a cell's RIP2 activity both in the presence and absence of the candidate compound, wherein a decreased activity of RIP2 in the presence of the compound indicates that the compound is a RIP2 inhibitor. In one embodiment of this method, the compound and the cells are contacted under conditions permitting entry of the compound into the cell.

This invention provides a method of producing an anti-inflammatory effect and an immuno-inhibitory effect in an individual, comprising administering to the individual a compound that inhibits RIP2 in sufficient quantity to inhibit RIP2, thereby producing an anti-inflammatory effect and an immuno-inhibitory effect in the individual.

As used herein, a “individual” refers to a mammal, including but not limited to a mouse, a hamster, a rat, a goat, a rabbit, a primate, a dog, or a human. In another embodiment, the subject suffers from an inflammatory disease. In one embodiment, the subject suffers from an autoimmune condition such as rheumatoid arthritis or lupus erythematosus.

This invention provides a method of treating an inflammatory condition in an individual comprising administering to the individual a compound that inhibits RIP2 activity in the individual, thereby producing an anti-inflammatory effect in the individual. In one embodiment, the inflammatory condition is an autoimmune condition. In one embodiment, the autoimmune condition is rheumatoid arthritis or lupus erythematosus.

In one embodiment of this method, use of a compound which inhibits RIP2 for the preparation of a medicament which provides an anti-inflammatory effect and immuno-inhibitory effect in an individual. In one embodiment of this method, use of a compound which inhibits RIP2 for the preparation of a medicament for treating an inflammatory condition in an individual.

This invention provides a method of determining whether a compound is a RIP2 inhibitor comprising: (a) contacting a cell expressing RIP2 with a candidate compound and measuring the cell's production of an inflammatory cytokine or chemokine upon stimulation with a TLR ligand; (b) comparing the cell's production of the inflammatory cytokine or chemokine in step (a) with the cell's production of the inflammatory cytokine or chemokine in the absence of the candidate compound; (c) contacting a cell which does not express RIP2 with the candidate compound and measuring the cell's production of an inflammatory cytokine or chemokine upon stimulation with a TLR ligand; and (d) comparing the cell's production of the inflammatory cytokine or chemokine in step (c) with the cell's production of the inflammatory cytokine or chemokine in the absence of the candidate compound; wherein the production measured in (a) is less than the production measured in (b), and the production measured in step (c) is comparable to the production measured in step (d) indicates that the compound is a RIP2 inhibitor. The meaning of “comparable” as used herein maybe explained with reference to the above embodiment. For example, amount measured in step (c) is comparable to the amount measured in step (d) if the difference in magnitude between the amount measured in step (c) and the amount measured in step (d) is less than the difference in magnitude between the amount measured in step (a) and the amount measured in step (b).

The steps recited in the above method may be performed in any order. The invention is not limited to a method which recites the steps in the order provided above.

In one embodiment, the TLR ligand is capable of decreasing production of an inflammatory cytokine. In one embodiment, the TLR ligand is a TLR4 ligand or a TLR2 ligand. In one embodiment, the TLR ligand is TLR4 ligand, and wherein the TLR4 ligand is LPS or lipoteichoic acid (“LTA”). In one embodiment, the TLR ligand is a TLR2 ligand, and wherein the TLR2 ligand is peptidoglycan. In one embodiment, the inflammatory cytokine is IL-6 or TNF-α. In one embodiment, the chemokine is IP10.

This invention provides a method of determining whether a compound is a RIP2 inhibitor comprising: (a) contacting a cell expressing RIP2 with a candidate compound and measuring the cell's production of an inflammatory cytokine or chemokine upon stimulation with a pathogen; (b) comparing the cell's production of the inflammatory cytokine or chemokine of step (a) with the cell's production of the inflammatory cytokine or chemokine in the absence of the candidate compound; (c) contacting a cell which does not express RIP2 with the candidate compound and measuring the cell's production of an inflammatory cytokine or chemokine upon stimulation with a pathogen; and (f) comparing the cell's production of the inflammatory cytokine or chemokine in step (c) with the cell's production of the inflammatory cytokine or chemokine in the absence of the candidate compound; wherein the production measured in (a) is less than the production measured in (b), and the production measured in step (c) is comparable to the production measured in step (d) indicates that the compound is a RIP2 inhibitor.

The steps recited in the above method may be performed in any order. The invention is not limited to a method which recites the steps in the order provided above.

In one embodiment, the contacting is conducted under conditions appropriate for entry of the candidate compounds into the cells. In one embodiment, the pathogen is Listeria monocytogenes. In one embodiment, the inflammatory cytokine is IL-6 or TNF-α.

This invention provides a method of determining whether a compound is a RIP2 inhibitor comprising: (a) contacting a cell expressing RIP2 with a candidate compound and measuring NF-κB activation in the cell; (b) comparing the NF-κB activation measured in step (a) with the activation of NF-κB measured in a cell expressing RIP2 in the absence of the candidate compound; (c) contacting a cell which does not express RIP2 with the candidate compound and measuring the activation of NF-κB in the cell; and (d) comparing the NF-κB activation measured in step (c) with the NF-κB activation measured in a cell which does not express RIP2 in the absence of the candidate compound; wherein the activator measured in (a) is less than the activation measured in (b), and the activation measured in step (c) is comparable to the activation measured in step (d) indicates that the compound is a RIP2 inhibitor.

The steps recited in the above method may be performed in any order. The invention is not limited to a method which recites the steps in the order provided above.

In one embodiment, the contacting is conducted under conditions appropriate for entry of the candidate compound into the cells. In one embodiment, the NF-κB activation is decreased upon T cell receptor stimulation. In one embodiment, the NF-κB activation is determined by examining the phosphorylation state of an NF-κB substrate. The NF-κB substrates include but are not limited to p38, IαBα, ERK or JNK. In one embodiment, Nod1 and/or Nod2 effects the NF-κB activation. In one emboediment, NF-κB activation is detected by measuring IκBα degradation. In one embodiment, NF-κB activation is measured by gel shift assay.

This invention provides a method of determining whether a compound is a RIP2 inhibitor comprising: (a) contacting a cell expressing RIP2 with a candidate compound and measuring cell proliferation, upon stimulation with IL-2 or Concanavalin A, alone or together with IL-1β; (b) comparing the cell proliferation in step (a) with the proliferation of a cell expressing RIP2 in the absence of the candidate compound, upon stimulation with IL-2 or Concanavalin A, alone or together with IL-1β; (c) contacting a cell which does not express RIP2 with the candidate compound and measuring cell proliferation, upon stimulation with IL-2 or Concanavalin A, alone or together with IL-1β; and (d) comparing the cell proliferation in step (c) with the proliferation of a cell which does not express RIP2 in the absence of the candidate compound upon stimulation with IL-2 or Concanavalin A, alone or in combination with IL-1β; wherein cell proliferation measured in step (a) is less than in step (b), and cell proliferation in step (d) is comparable to step (a) indicates that the compound is a RIP2 inhibitor.

The steps recited in the above method may be performed in any order. The invention is not limited to a method which recites the steps in the order provided above.

In one embodiment, the contacting is conducted under conditions appropriate for entry of the candidate compounds into the cells.

This invention provides a method of determining whether a compound is a RIP2 inhibitor comprising: (a) contacting a cell expressing RIP2 with a candidate compound and measuring the amount of the cell's IL-2 production upon T cell receptor stimulation; (b) comparing the amount of IL-2 measured in step (a) with an amount of the cell's IL-2 production in the absence of the candidate compound upon T cell receptor stimulation; (c) contacting a cell which does not express RIP2 with the candidate compound and measuring the cell's IL-2 production upon T cell receptor stimulation; and (d) comparing the amount of IL-2 measured in step (c) with an amount of IL-2 produced by a cell that does not express RIP2 in the absence of the candidate compound upon T cell receptor stimulation; wherein an amount measured in step (a) is less than step (b), and an amount measured in step (c) is comparable to the amount measured in step (d) indicates that the compound is a RIP2 inhibitor.

The steps recited in the above method may be performed in any order. The invention is not limited to a method which recites the steps in the order provided above.

In one embodiment, the contacting is conducted under conditions appropriate for entry of the candidate compounds into the cells.

This invention provides a method of determining whether a compound is a RIP2 inhibitor comprising: (a) contacting cells expressing RIP2 with a candidate compound and measuring proliferation of the cells upon T cell receptor stimulation; (b) comparing the amount of proliferation measured in step (a) with the proliferation of cells expressing RIP2 in the absence of the candidate compound upon T cell receptor stimulation; (c) contacting cells which do not express RIP2 with the candidate compound and measuring the proliferation of the cells upon T cell receptor stimulation; (d) comparing the amount of proliferation measured in step (c) with the proliferation of cells which do not express RIP2 in the absence of the candidate compound upon T cell receptor stimulation; wherein an amount measured in step (a) is less than step (b), and an amount measured in step (c) is comparable to the amount measured in step (d) indicates that the compound is a RIP2 inhibitor.

The steps recited in the above method may be performed in any order. The invention is not limited to a method which recites the steps in the order provided above. In one embodiment, the cell is a T cell. This invention provides an isolated cell which does not express RIP2. In one embodiment, the isolated cell is a mammalian cell. In one embodiment, the isolated cell is a fibroblast, T cell or macrophage. In one embodiment, the isolated cell is a mouse or human cell.

This invention provides an isolated cell which does not normally express RIP2, which comprises an exogeneous nucleic acid encoding RIP2. In one embodiment, the nucleic acid is contained in an expression vector. In one embodiment, the isolated cell obtained from a RIP2 deficient transgenic non-human animal. In one embodiment, the transgenic non-human animal is a mouse. In one embodiment, the transgenic non-human animal is a homozygous RIP2 deficient transgenic non-human animal.

The steps recited in the above method may be performed in any order. The invention is not limited to a method which recites the steps in the order provided above.

This invention provides a method of obtaining a composition which comprises: (a) identifying a compound by one of the methods described herein; (b) admixing the compound so identified or a homolog or derivative thereof with a carrier, so as to thereby obtain a composition. This invention provides compounds identified by the any of the methods described herein.

### EXAMPLE 1

Assessment of the role of RIP2 in signaling through proteins of the innate immune systems. Taken together, the results described in this example indicate that RIP2 is essential for signaling through TLRs and NOD protein family members, which are central components of the innate immune system.

A) TLRs

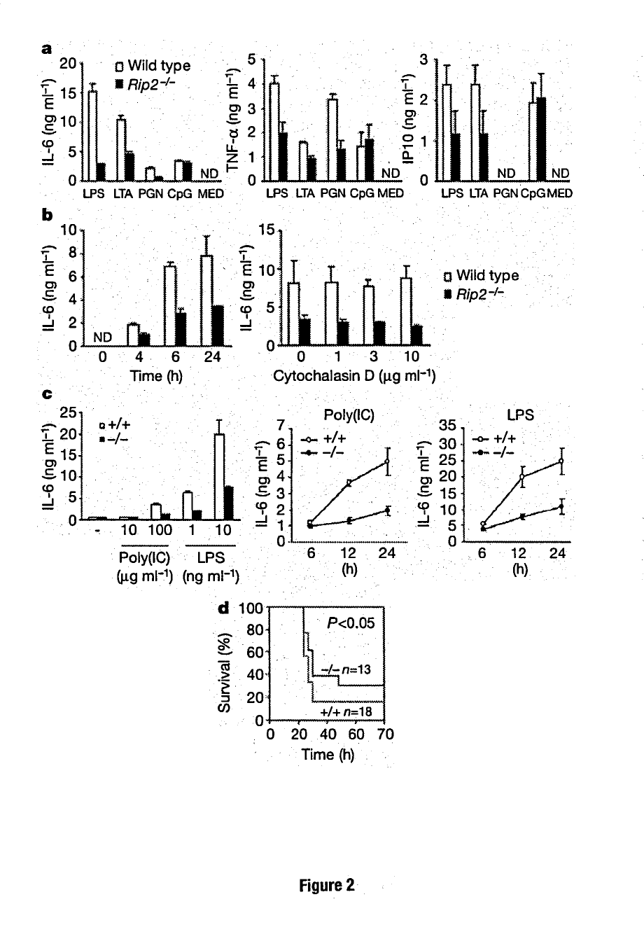

TLRs can recognize specific pathogen associated molecular patterns (PAMPs) such as LPS ⁶⁻⁸, lipoteichoic acid ⁹, peptidoglycan ⁹, CpG containing DNA ¹⁰, Flagellin ¹¹ or double-stranded RNA ¹². To test whether RIP2 is involved in TLR signaling, RIP2^{-/-} macrophages were stimulated with various PAMPs and cytokine/chemokine production was assessed by ELISA. Production of the inflammatory cytokines IL-6 and TNFα and the chemokine IP10 was severely reduced in RIP2^{-/-} macrophages upon stimulation with LPS (a ligand for TLR4), lipoteichoic acid (for TLR4), or peptidoglycan (for TLR2) (FIG. 2a). There was no defect in cytokine/chemokine production following stimulation with CpG DNA (a ligand for TLR9), indicating that RIP2 is required for TLR4 and TLR2 but not involved in TLR9 signaling. Production of IL-6 is also reduced in RIP2^{-/-} embryonic fibroblasts upon stimulation with double-stranded RNA, poly(IC) (a ligand for TLR3) and LPS in dose and time dependent manner (FIG. 2c). These results indicate that RIP2 is required for signaling through some but not all TLRs. To assess the response to a live pathogen, RIP2^{-/-} macrophages were infected with Listeria monocytogenes and the levels of IL-6 and TNFα were measured by ELISA.RIP2^{-/-} macrophages were compromised in their ability to produce these cytokines, further supporting the involvement of this kinase in the innate immune response (FIG. 2b) Phagocytosis of L. monocytogenes by macrophages can be inhibited by the actin-depolymerizing agent cytochalasin D ¹³. To examine the mechanism of activation of RIP2 in Listeria infected macrophages, in particular to determine whether Listeria stimulation of the innate immune response occurred at the cell surface or intracellularly, cytochalasin D was added to cell cultures at various concentrations since it blocks Listeria internalization ¹³. IL-6 production by Listeria infection was not altered even at high concentrations of cytochalasin D and in all cases was reduced in RIP₂^{-/-} cells (FIG. 2b). Thus, attachment of bacteria to the cell surface is sufficient to activate macrophages and the reduced cytokine production in RIP2^{-/-} cells is due to defective signaling from cell surface receptors. In vivo response to LPS by RIP2-deficient mice were assessed by endotoxin shock experiments using intraperitoneal injection of LPS. RIP2-deficient mice were more resistant to LPS than wild-type mice (FIG. 2d) suggesting the importance of this molecule in LPS response in vivo.

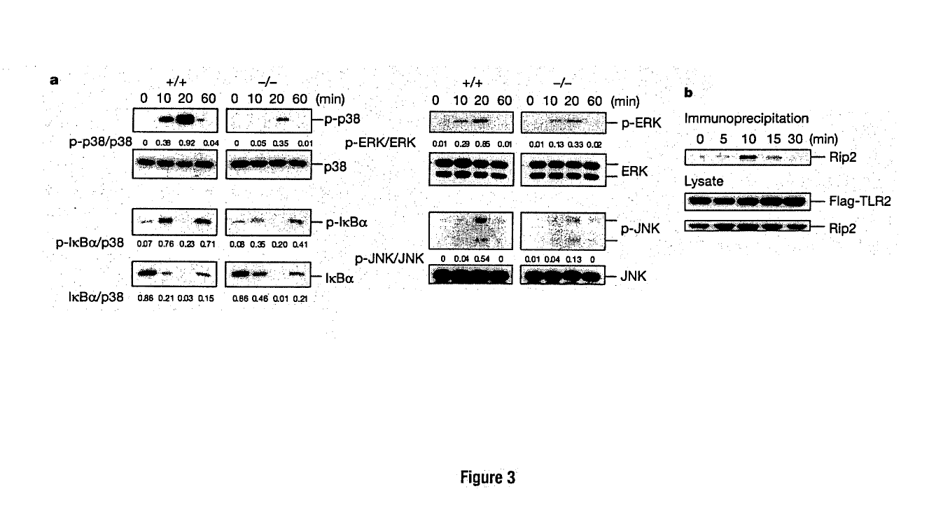

TLR signaling requires the formation of multiprotein signaling complexes, which include the serine/threonine kinase IRAK and the adapter molecules MyD88 and TRAF6 ^{14,15}. Applicants therefore tested if RIP2 can associate with TLR and TLR-associated signaling molecules. Cotransfection with vectors expressing Myc-tagged TLR2 or TLR4 together with a vector expressing Flag-tagged RIP2 demonstrated an association of RIP2 with TLR2 and TLR4 (FIG. 3a). Cotransfection with Myc-tagged TLR4 and RIP2 together with Flag-tagged MyD88 resulted in the association of MyD88 with TLR4 and RIP2 (FIG. 3b). These results suggest that RIP2 can associate with TLR signaling complexes either directly or indirectly.

Since TLR signaling results in the activation of NF-κB and the mitogen activated kinases (MAP) JNK, p38 and ERK1/2 ^{14,15}, Applicants studied activation of these molecules in RIP2^{-/-} macrophages by examining their phosphorylation state. LPS stimulated RIP2^{-/-} macrophages showed reduced levels of phosphorylation of p38, IκBα, ERK and JNK and reduced degradation of IκBA (FIG. 3c), indicating altered signaling downstream from TLR4. This reduced signaling was not due to changes in the expression levels of MyD88, IRAK or TRAF6, since Western blot analysis for these proteins showed no difference between wild type and RIP2^{-/-} macrophages.

B) Nod Proteins

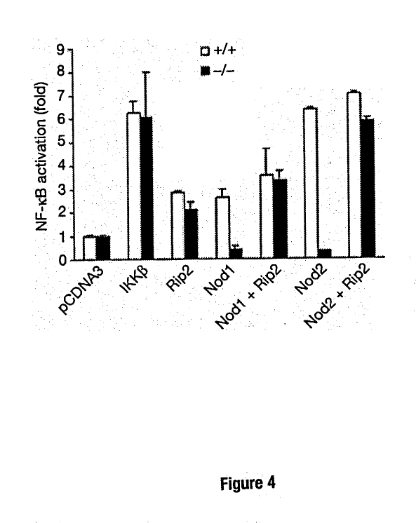

In addition to TLRs, increasing evidence implicates another family of proteins in innate immune responses. This family of cytoplasmic proteins, collectively termed Nod, is characterized by the presence of three motifs: a CARD,—an NBD (nucleotide binding domain) and—an LRR. These proteins have homology to the NBD-LRR type disease resistant gene products in plants ¹⁶⁻¹⁸. An increasing number of the members of this family have been identified (Nod1/CARD4, Nod2, DEFCAP/NAC, CARD12/Ipaf/CLAN) ¹⁶⁻²³ and by analogy to the plant molecules these data imply that, like the TLR family, Nod proteins are a diverse family of molecules designed to detect pathogens in intracellular compartments; the LRR of members of both families is likely to confer pathogen specificity ^{24,25}. In fact, Nod1 is activated upon infection of Shigella flexneri in epithelial cells ²⁶ and one NBD-LRR protein, NAIP determines susceptibility to Legionella pneumophila infection ²⁷. Also, it has been recently demonstrated that Nod2 is mutated in patients susceptible to Crohn's disease and Blau syndrome ^{25,28,29}. In vitro studies showed that Nod1 and Nod2 bind to RIP2 via a CARD/CARD interaction ^{17,18}, suggesting that RIP2 may be involved in signaling downstream of the Nod family proteins. To test this, Applicants generated RIP2-deficient embryonic fibroblasts and cotransfected them with both Nod expression vectors and an NF-κB reporter construct. NF-κB activation by Nod1/Nod2 expression was completely abolished in RIP2^{-/-} fibroblasts and complementation of RIP2^{-/-} fibroblasts with a RIP2 expression vector restored these defects (FIG. 4). Cotransfection of dominant negative MyD88, which abrogates TLR signaling, together with a Nod1 expression vector resulted in a strong response to LPS in wild type fibroblasts, but not in RIP2^{-/-} fibroblasts suggesting that Nod1 increases the sensitivity to LPS independently of TLRs as previously shown in 293T cells ²⁴. There results indicate in this example, results indicate that RIP2 is essential for signaling through TLRs and Nod protein family members, which are central components of the innate immune system.

### EXAMPLE 2

Assessment of the role of RIP2 in signaling the adaptive immune system results described indicate that RIP2 is required for appropriate T cell receptor (TCR) signaling:

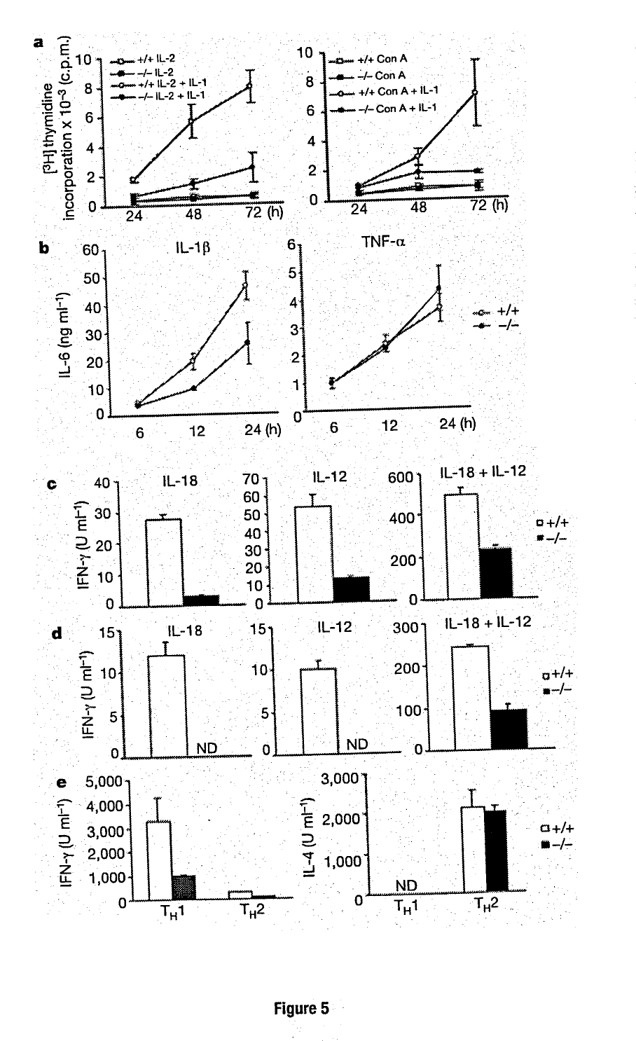

IL-1 and IL-18 receptors both have TIR (Toll/IL1-receptor) domain within the cytoplasmic tail and therefore belong to the same multi-gene family as the TLRs. Applicants therefore tested to determine whether responses to IL-1 or IL-18 were altered in RIP2^{-/-} cells. Under certain conditions IL-1β is a potent costimulant to T cells for their growth. Thymocytes were stimulated with IL-1β, together with IL-2 or low dose Concanavalin A (ConA). RIP2^{-/-} thymocytes showed reduced proliferation upon IL-1β, stimulation in both IL-2 and Con A costimulation (FIG. 5a). Embryonic fibroblasts produce IL-6 upon stimulation with the cytokines IL-1β or TNFα. RIP2^{-/-} embryonic fibroblasts were significantly impaired in their ability to produce IL-6 when stimulated with IL-1β, but not when stimulated with TNFα suggesting that RIP2 is involved in IL-1- but not TNFα-receptor signaling (FIG. 5b). Next, Applicants investigated the IL-18 response in RIP2^{-/-} cells. IFNγ production by NK cells upon IL-18 stimulation was assessed using RIP2^{-/-} splenocytes. IFNγ production was severely reduced in RIP2^{-/-} cells by IL-18 stimulation (FIG. 5c). Surprisingly, IFNγ production upon IL-12 stimulation was also reduced. Costimulation of RIP2^{-/-} cells with IL-18 and I1-12 also resulted in reduced production of IFNγ (FIG. 5c) and IL-12 response was examined using differentiated effector CD4⁺Th1 cells. It has been shown that, similar to NK cells, effector Th1 cells make copious amounts of IFNγ when stimulated with IL-18 and IL-12 in the absence of T cell receptor (TCR) stimulation ³⁰ IFNγ production by RIP2^{-/-} Th1 cells with IL-18, IL12 or a combination of IL-18 and IL-12 stimulation was severely perturbed (FIG. 5d). These results support the conclusion that RIP2 is involved in signaling downstream of the TLR/IL-1 receptor family and the altered IL-12 response indicates that RIP2 may be involved in IL-12 signaling either directly or indirectly. IL-12 is one of the key cytokines regulating T cell differentiation. Therefore, T cell differentiation of RIP2 ^{-/-} T cells was analyzed. CD4⁺T cells were cultured for 4 days under either Th1 or Th2 conditions and restimulated with plate-bound anti-CD3 antibodies for 24 hours. IFNγ production by RIP2^{-/-} Th1 cells was reduced, although IL-4 production of RIP2^{-/-} Th2 cells was not affected. These results suggest that RIP2 plays an important role in the differentiation of Th1 cells but not Th2 cells.

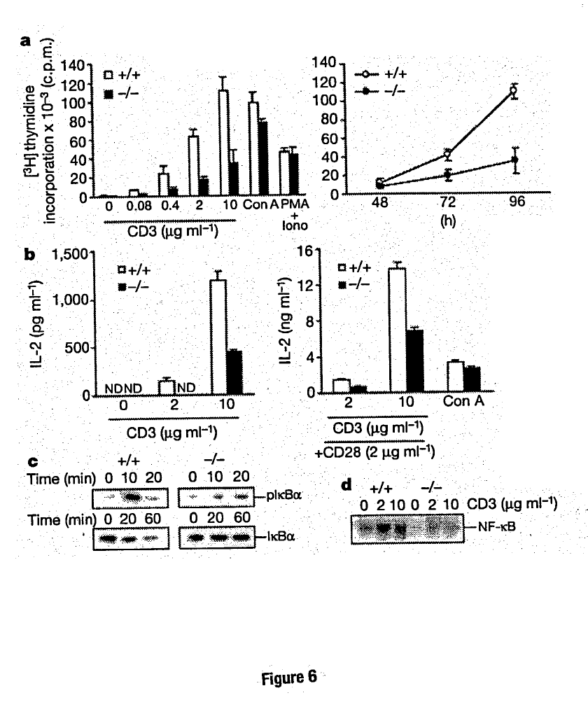

It has been previously shown that altered TCR signaling has a profound influence on Th1/Th2 differentiation ³¹. The response of RIP2^{-/-} T cells upon TCR stimulation with anti-CD3 antibodies was examined. RIP2^{-/-} CD4⁺T cells showed severely reduced proliferation upon anti-CD3 stimulation in a dose and time dependent manner (FIG. 6a). IL-2 production was reduced in RIP2^{-/-} CD4⁺T cells and this defect could not be rescued by costimulation with anti-CD28 (FIG. 6b). Since RIP2 is involved in NF-κB activation ¹⁻⁴ and a prior study showed NF-κB activation is required for T cell proliferation upon TCR stimulation ^{32,33}, Applicants analyzed NF-κB activation in RIP2^{-/-} T cells upon anti-CD3 stimulation. Phosphorylation of IκBα and degradation of IκBα assessed by Western blotting was reduced in RIP2^{-/-} T cells (FIG. 6c) and NF-κB activation assessed by gel-shift assay was also substantially reduced (FIG. 6d). Taken together, these data indicated that RIP2 is required for optimal activation of NF-κB and T cell proliferation upon TCR stimulation. Since inhibition of NF-κB activation can cause Th1 deficiency in vivo ³⁴, the Th1 deficiency in RIP2^{-/-} T cells may be attributable, at least in part, to altered TCR signaling and NF-κB activation.

### MATERIALS AND METHODS

The following materials and methods were used in the work described herein.

Generation of RIP2-deficient mice. A murine RIP2-encoding partial cDNA was obtained by PCR using mouse heart first strand cDNA (CLONTECH) as the template and a specific primer based on GenBank accession no. AA655189 (reverse, 5′-TCA TTA TCC AAC AAG ATA TTC TGA GTC T) and a primer based on the human RIP2 sequence (forward, 5′-GAG GCC ATC TGC AGC GCC CTG CCC AC). The 430 bp cDNA obtained was subcloned into the T-Vector (Promega) and its identity verified by sequence analysis. 129SV/J genomic library (Stratagene) was screened with the murine RIP2 cDNA to obtain a mouse RIP2 genomic clone. Two phage carrying overlapping genomic clones encompassing RIP2 were isolated. A targeting vector was designed to replace a 4.0 kb genomic fragment containing the 2^{nd} and 3^{rd} exons encoding the active site aspartate residue with the loxP-flanked neomycin resistance (neo-) gene expression cassette. The targeting vector was linearized with NotI and electroporated into W9.5 ES cells. Clones resistant to G418 and gancyclovir were selected, and homologous recombination was confirmed by Southern blotting. Six out of 135 clones screened were positive for homologous recombination. Three clones homologous for the targeted mutation were injected into C57BL/6 blastocysts, which were subsequently transferred into pseudopregnant foster mothers. The resulting male chimeric mice were bred to C57BL/6 females to obtain heterozygous mice. Gernline transmission of the mutant allele was verified by Southern blot analysis of tail DNA from F1 offspring with agouti coat color. Interbreeding of the obtained heterozygous mice was performed to generate homozygous RIP2-deficient mice.

Plasmids: The expression vectors pcDNA3-IKKβ, pcDNA3-Myc-RIP2, pcDNA3-Nod1-Flag, pcDNA3-Nod2-Flag, pEF1-BOS-β-gal, pBVI-Luc, pcDNA3-DN-MyD88, pcDNA3-TLR2ΔLRR-Myc, pcDNA3-TLR4ΔLRR-Myc, pCMV-Flag-RIP2, pcDNA3-Flag-MyD88 were described previously ^{18,35}.

Reagents: Lipopolysacchride (LPS) from Salmonella abortus equi and lipoteichoic acid (LTA) from Staphylococcus aureus were purchased from Sigma. Peptidoglycan (PGN) from Staphylococcus aureus was from Fluka. Poly(IC) was from Amersham Pharmacia Biotech. Phosphorothioate-modified CpG oligo DNA (tccatgacgttcctgacgtt) was synthesized in HHMI Biopolymer & W. M. Keck Biotechnology Resource Laboratory in Yale University. Human IL-1β and mouse TNFα were from R&D. Mouse IL-18 was from MBL. Mouse IL-12 was from Gentetic Institute. Mouse IL-4 was from PharMingen. Anti-CD3, anti-IL-4 or anti-IFNγ were purified from supernatants of 2C11, 11B11 or XMG hybridoma respectively.

Culture of Bone marrow derived macrophages: Bone marrow derived macrophages were prepared as described before ³⁶. Cells were harvested with cold DPBS, washed, resuspended in DMEM supplemented with 10% of Fetal calf serum and used at a density of 2×10⁵/ml in the experiments. Cells were left untreated for at least 4 h at 37° C. in 10% CO₂ prior to further handling.

Listeria infection of macrophages: The cells were cultured without antibiotics and listeriae (ATCC strain 43251) were added at an MOI of 50. After incubation for 30 min, extracellular bacteria were removed by washing three times with DPBS. To prevent reinfection, the cells were cultured in medium containing gentamicin sufate (50 μg/ml, GIBCO BRL) measurement of cytokine production from macrophages and embryonic fibroblasts. Bone marrow derived macrophages were cultured with the indicated concentration of LPS, LTA, PGN or CpG DNA for 6 h. Embryonic fibroblasts were cultured with poly(IC), LPS, IL-1β or TNFα at the indicated concentration for the indicated periods. The concentration of IL-6, TNF-α and IP10 in culture supernatants was measured by ELISA.

LPS endotoxin shock in vivo: Mice were injected with 16.7 mg/kg body weight LPS from Salmonella abortus equi. Animals were observed every 12 hours for 5 days and time of death and mortality were recorded.

Proliferation Assays: CD4⁺ T cells were purified as described before³⁷.Thymocytes were stimulated with IL-2 (2 ng/ml) or Con A (0.625 μg/ml) in the presence or absence of IL-1β (10 ng/ml). CD4⁺ T cells were stimulated with anti-CD3 (2C11) in the presence of T cell depleted irradiated splenocytes. Cells were pulsed with [³H] thymidine for 8 hours and its incorporation was measured by with a β plate counter (Wallac).

Cytokine production by NK cells and T cells: Th1/Th2 differentiated cells were washed and restimulated with 10 μg/ml of anti-CD3 and cultured for 24 hours. IFNγ. and IL-4 in the supernatant was measured. Total splenocytes and Th1 cells were stimulated with IL-18 (10 ng/ml), IL-12 (10 ng/ml) or a combination of both. The concentration of IFNγ and IL-4 was measured by ELISA. For IL-2 production, purified CD4⁺ T cells were stimulated with plate-bound anti-CD3 in the presence or absence of anti-CD28 (2 μg/ml) at the indicated concentration for 24 hours and the level of IL-2 in the supernatant was measured by ELISA.

NF-κB activation assay: The NF-κB activation assays were carried out as described ¹. Briefly, mouse embryonic fibroblasts were co-transfected with 12 ng of the reporter construct pBVI-Luc, the indicated expression plasmids and 120 ng of pEF-BOS-β-gal. Twenty-four hours after transfection, cell extracts were prepared and the relative luciferase activity was measured as described. Results were normalized for transfection efficiency with values obtained with pEF-BOS-β-gal.

Western Blot Analysis and Immunoprecipitation: Cell lysis and blotting were carried out as described ³⁸. Membranes were blotted with antibodies to RIP2 (Cayman), phosphorylated-IκB, IκB, phosphorylated-JNK, JNK, phosphorylated-p38, p38, phosphorylated-ERK1/2, ERK1/2 (Cell signaling), IRAK-1, TRAF6 (Santa Cruz) and MyD88 (StressGen). Immunoprecipitation was carried out as described before ³⁸ using an anti-FLAG monoclonal antibody (Sigma) and coimmunoprecipitated proteins were detected with a polyclonal anti-Myc antibody (Santa Cruz).

Northern Blot Analysis: Bone Marrow derived macrophages were stimulated with 10 ng/ml of LPS for the indicated periods. Preparation of total RNA samples and Northern blot analysis were performed as described before³⁸.

T cell stimulation for NF-κB activation: Purified splenic T cells were incubated with anti-CD3 antibodies at the indicated concentration for 30 min on ice. After washing, cells were incubated with anti-hamster IgG antibody (100 μg/ml, Vector) at 37° C. for the indicated periods. Cytoplasmic and nuclear extracts were used for Western Blot Analysis and Gel Mobility Shift Assay using an NF-κB specific probe, respectively.

### REFERENCES

1. Inohara, N., del Peso, L., Koseki, T., Chen, S. & Nunez, G. RICK, a novel protein kinase containing a caspase recruitment domain, interacts with CLARP and regulates CD95-mediated apoptosis. J Biol Chem 273, 12296-300. (1998).

2. McCarthy, J. V., Ni, J. & Dixit, V. M. RIP2 is a novel NF-kappaB-activating and cell death-inducing kinase. J Biol Chem 273, 16968-75. (1998).

3. Thome, M. et al. Identification of CARDIAK, a RIP-like kinase that associates with caspase-1. Curr Biol 8, 885-8. (1998).

4. Medzhitov, R. & Janeway, C., Jr. Innate immune recognition: mechanisms and pathways. Immunol Rev 173, 89-97. (2000).

5. Kelliher, M. A. et al. The death domain kinase RIP mediates the TNF-induced NF-kappaB signal. Immunity 8, 297-303. (1998).

6. Poltorak, A. et al. Defective LPS signaling in C3H/HeJ and C57BL/10ScCr mice: mutations in Tlr4 gene. Science 282, 2085-8. (1998).

7. Hoshino, K. et al. Cutting edge: Toll-like receptor 4 (TLR4)-deficient mice are hyporesponsive to lipopolysaccharide: evidence for TLR4 as the Lps gene product. J Immunol 162, 3749-52. (1999).

8. Qureshi, S. T. et al. Endotoxin-tolerant mice have mutations in Toll-like receptor 4 (Tlr4). J Exp Med 189, 615-25. (1999).

9. Takeuchi, O. et al. Differential roles of TLR2 and TLR4 in recognition of gram-negative and gram-positive bacterial cell wall components. Immunity 11,443-51. (1999).

10. Hemmi, H. et al. A Toll-like receptor recognizes bacterial DNA. Nature 408, 740-5. (2000).

11. Hayashi, F. et al. The innate immune response to bacterial flagellin is mediated by Toll-like receptor 5. Nature 410, 1099-103. (2001).

12. Alexopoulou, L., Holt, A. C., Medzhitov, R. & Flavell, R. A. Recognition of double-stranded RNA and activation of NF-kappaB by Toll- like receptor 3. Nature 413, 732-8. (2001).

13. Demuth, A., Goebel, W., Beuscher, H. U. & Kuhn, M. Differential regulation of cytokine and cytokine receptor mRNA expression upon infection of bone marrow-derived macrophages with Listeria monocytogenes. Infect Immun 64, 3475-83. (1996).

14. Medzhitov, R. et al. MyD88 is an adaptor protein in the hToll/IL-1 receptor family signaling pathways. Mol Cell 2, 253-8. (1998).

15. Kawai, T., Adachi, O., Ogawa, T., Takeda, K. & Akira, S. Unresponsiveness of MyD88-deficient mice to endotoxin. Immunity 11, 115-22. (1999).

16. Bertin, J. et al. Human CARD4 protein is a novel CED-4/Apaf-1 cell death family member that activates NF-kappaB. J Biol Chem 274, 12955-8. (1999).

17. Inohara, N. et al. Nod1, an Apaf-1-like activator of caspase-9 and nuclear factor-kappaB. J Biol Chem 274, 14560-7. (1999).

18. Ogura, Y. et al. Nod2, a Nod1/Apaf-1 family member that is restricted to monocytes and activates NF-kappaB. J Biol Chem 276, 4812-8. (2001).

19. Hlaing, T. et al. Molecular cloning and characterization of DEFCAP-L and -S, two isoforms of a novel member of the mammalian Ced-4 family of apoptosis proteins. J Biol Chem 276, 9230-8. (2001).

20. Geddes, B. J. et al. Human card12 is a novel ced4/apaf-1 family member that induces apoptosis. Biochem Biophys Res Commun 284, 77-82. (2001).

21. Chu, Z. L. et al. A novel enhancer of the Apaf1 apoptosome involved in cytochrome c- dependent caspase activation and apoptosis. J Biol Chem 276, 9239-45. (2001).

22. Poyet, J. L. et al. Identification Of Ipaf, A Human Caspase-1 Activating Protein Related To Apaf-1. J Biol Chem 4, 4 (2001).

23. Damiano, J. S., Stehlik, C., Pio, F., Godzik, A. & Reed, J. C. CLAN, a novel human CED-4-like gene. Genomics 75, 77-83. (2001).

24. Inohara, N., Ogura, Y., Chen, F. F., Muto, A. & Nunez, G. Human nod1 confers responsiveness to bacterial lipopolysaccharides. J Biol Chem 276, 2551-4. (2001).

25. Ogura, Y. et al. A frameshift mutation in NOD2 associated with susceptibility to Crohn's disease. Nature 411, 603-6. (2001).

26. Girardin, S. E. et al. CARD4/Nod1 mediates NF-kappaB and JNK activation by invasive Shigella flexneri. EMBO Rep 2, 736-42. (2001).

27. Growney, J. D. & Dietrich, W. F. High-resolution genetic and physical map of the Lgn1 interval in C57BL/6J implicates Naip2 or Naip5 in Legionella pneumophila pathogenesis. Genome Res 10, 1158-71. (2000).

28. Hugot, J. P. et al. Association of NOD2 leucine-rich repeat variants with susceptibility to Crohn's disease. Nature 411, 599-603. (2001).

29. Miceli-Richard, C. et al. CARD15 mutations in Blau syndrome. Nat Genet 29, 19-20. (2001).

30. Yang, J., Zhu, H., Murphy, T. L., Ouyang, W. & Murphy, K. M. IL-18-stimulated GADD45 beta required in cytokine-induced, but not TCR-induced, IFN-gamma production. Nat Immunol 2, 157-64. (2001).

31. Leitenberg, D. & Bottomly, K. Regulation of naive T cell differentiation by varying the potency of TCR signal transduction. Semin Immunol 11, 283-92. (1999).

32. Ruland, J. et al. Bc110 is a positive regulator of antigen receptor-induced activation of NF-kappaB and neural tube closure. Cell 104, 33-42. (2001).

33. Sun, Z. et al. PKC-theta is required for TCR-induced NF-kappaB activation in mature but not immature T lymphocytes. Nature 404, 402-7. (2000).

34. Aronica, M. A. et al. Preferential role for NF-kappa B/Rel signaling in the type 1 but not type 2 T cell-dependent immune response in vivo. J Immunol 163, 5116-24. (1999).

35. Horng, T., Barton, G. M. & Medzhitov, R. TIRAP: an adapter molecule in the Toll signaling pathway. Nat Immunol 2, 835-41. (2001).

36. Celada, A., Gray, P. W., Rinderknecht, E. & Schreiber, R. D. Evidence for a gamma-interferon receptor that regulates macrophage tumoricidal activity. J Exp Med 160, 55-74. (1984).

37. Zheng, W. & Flavell, R. A. The transcription factor GATA-3 is necessary and sufficient for Th2 cytokine gene expression in CD4 T cells. Cell 89, 587-96. (1997).

38. Kobayashi, K., Hatano, M., Otaki, M., Ogasawara, T. & Tokuhisa, T. Expression of a murine homologue of the inhibitor of apoptosis protein is related to cell proliferation. Proc Nati Acad Sci U S A 96, 1457-62. (1999).

What is claimed:

1. A method of identifying a compound that modulates an innate immune response and an adaptive immune response comprising contacting cells expressing RIP2 with a candidate compound, and determining whether the candidate compound modulates RIP2 activity in the cells, wherein modulation of RIP2 activity in the cells by the candidate compound indicates that the candidate compound modulates the innate immune response and the adaptive immune response.

2. The method of claim 1, wherein the contacting is conducted under conditions appropriate for entry of the candidate compound into the cells.

3. The method of claim 1, further comprising the step of comparing the RIP2 activity in the presence of the candidate compound with the RIP2 activity of a standard known to be deficient in RIP2 activity, wherein RIP2 activity in the presence of the candidate compound which is comparable to RIP2 activity of the known standard indicates that the compound is a RIP2 inhibitor.

4. The method of claim 3, when the standard is the RIP2 activity determined in a cell which does not express RIP2.

5. The method of claim 1, wherein the modulator is an inhibitor of RIP2 activity, and wherein inhibition of RIP2 activity in the cells by the candidate compound indicates that the candidate compound inhibits the innate immune response and adaptive immune response.

6. The method of claim 1, wherein the innate immune response is production of inflammatory cytokines and the adaptive immune response is production of antibodies.

7. A method of identifying a compound that produces an anti-inflammatory effect and an immuno-inhibitory effect comprising contacting cells expressing RIP2 with a candidate compound and determining whether the candidate compound inhibits RIP2 activity in the cells, wherein if inhibition of RIP2 activity by the candidate compound occurs compound that produces an anti-inflammatory effect and an immuno-inhibitory effect is identified.

8. The method of claim 7, wherein the contacting is conducted under conditions appropriate for entry of the candidate compounds into the cells.

9. The method of claim 7, further comprising the step of comparing the RIP2 activity in the presence of the candidate compound with the RIP2 activity for a standard known to be deficient in RIP2 activity, wherein RIP2 activity in the presence of the candidate compound which is comparable to RIP2 activity for the known standard indicates that the compound is a RIP2 inhibitor.

10. The method of claim 9, when the standard is the RIP2 activity determined in a cell which does not express RIP2.

11. A method of determining whether a compound is a RIP2 inhibitor comprising comparing a cell's RIP2 activity both in the presence and absence of the candidate compound, wherein a decreased activity of RIP2 in the presence of the compound indicates that the compound is a RIP2 inhibitor.

12. The method of claim 11, wherein the compound and the cells are contacted under conditions permitting entry of the compound into the cell.

13. A method of producing an anti-inflammatory effect and an immuno-inhibitory effect in an individual, comprising administering to the individual a compound that inhibits RIP2 in sufficient quantity to inhibit RIP2, thereby producing an anti-inflammatory effect and an immuno-inhibitory effect in the individual.

14. A method of treating an inflammatory condition in an individual comprising administering to the individual a compound that inhibits RIP2 activity in the individual, thereby producing an anti-inflammatory effect in the individual.

15. Use of a compound which inhibits RIP2 for the preparation of a medicament which provides an anti-inflammatory effect and immuno-inhibitory effect in an individual.

16. Use of a compound which inhibits RIP2 for the preparation of a medicament for treating an inflammatory condition in an individual.

17. The method of claim 14, wherein the inflammatory condition is an autoimmune condition.

18. The method of claim 7, wherein the autoimmune condition is rheumatoid arthritis or lupus erythematosus.

19. A method of determining whether a compound is a RIP2 inhibitor comprising:
a. contacting a cell expressing RIP2 with a candidate compound and measuring the cell's production of an inflammatory cytokine or chemokine upon stimulation with a TLR ligand;
b. comparing the cell's production of the inflammatory cytokine or chemokine in step (a) with the cell's production of the inflammatory cytokine or chemokine in the absence of the candidate compound;
c. contacting a cell which does not express RIP2 with the candidate compound and measuring the cell's production of an inflammatory cytokine or chemokine upon stimulation with a TLR ligand; and
d. comparing the cell's production of the inflammatory cytokine or chemokine in step (c) with the cell's production of the inflammatory cytokine or chemokine in the absence of the candidate compound;
wherein the production measured in (a) is less than the production measured in (b), and the production measured in step (c) is comparable to the production measured in step (d) indicates that the compound is a RIP2 inhibitor.

20. The method of claim 19, wherein the TLR ligand is capable of decreasing production of an inflammatory cytokine.

21. The method of claim 20, wherein the TLR ligand is a TLR4 ligand or a TLR2 ligand.

22. The method of claim 21, wherein the TLR ligand is TLR4 ligand, and wherein the TLR4 ligand is LPS or lipoteichoic acid (“LTA”).

23. The method of claim 21 wherein the TLR ligand is a TLR2 ligand, and wherein the TLR2 ligand is peptidoglycan.

24. The method of claim 19, wherein the inflammatory cytokine is IL-6 or TNF-α.

25. The method of claim 19, wherein the chemokine is IP10.

26. A method of determining whether a compound is a RIP2 inhibitor comprising:
a. contacting a cell expressing RIP2 with a candidate compound and measuring the cell's production of an inflammatory cytokine or chemokine upon stimulation with a pathogen;
b. comparing the cell's production of the inflammatory cytokine or chemokine of step (a) with the cell's production of the inflammatory cytokine or chemokine in the absence of the candidate compound;
c. contacting a cell which does not express RIP2 with the candidate compound and measuring the cell's production of an inflammatory cytokine or chemokine upon stimulation with a pathogen; and
d. comparing the cell's production of the inflammatory cytokine or chemokine in step (c) with the cell's production of the inflammatory cytokine or chemokine in the absence of the candidate compound;
wherein the production measured in (a) is less than the production measured in (b), and the production measured in step (c) is comparable to the production measured in step (d) indicates that the compound is a RIP2 inhibitor.

27. The method of claim 26, wherein the contacting is conducted under conditions appropriate for entry of the candidate compounds into the cells.

28. The method of claim 26, wherein the pathogen is Listeria monocytogenes.

29. The method of claim 19 or 26, wherein the inflammatory cytokine is IL-6 or TNF-α.

30. A method of determining whether a compound is a RIP2 inhibitor comprising:
a. contacting a cell expressing RIP2 with a candidate compound and measuring NF-κB activation in the cell;
b. comparing the NF-κB activation measured in step (a) with the activation of NF-κB measured in a cell expressing RIP2 in the absence of the candidate compound;
c. contacting a cell which does not express RIP2 with the candidate compound and measuring the activation of NF-κB in the cell; and
d. comparing the NF-κB activation measured in step (c) with the NF-κB activation measured in a cell which does not express RIP2 in the absence of the candidate compound;
wherein the activator measured in (a) is less than the activation measured in (b), and the activation measured in step (c) is comparable to the activation measured in step (d) indicates that the compound is a RIP2 inhibitor.

31. The method of claim 30, wherein the contacting is conducted under conditions appropriate for entry of the candidate compound into the cells.

32. The method of claim 30, wherein the NF-κB activation is decreased upon T cell receptor stimulation.

33. The method of claim 30, wherein the NF-κB activation is determined by examining the phosphorylation state of an NF-κB substrate.

34. The method of claim 30, wherein Nod1 and/or Nod2 effects the NF-κB activation.

35. The method of claim 30, wherein NF-κB activation is detected by measuring IκBα degradation.

36. The method of claim 30, wherein NF-κB activation is measured by gel shift assay.

37. A method of determining whether a compound is a RIP2 inhibitor comprising:
a. contacting a cell expressing RIP2 with a candidate compound and measuring cell proliferation, upon stimulation with IL-2 or Concanavalin A, alone or together with IL-1β;
b. comparing the cell proliferation in step (a) with the proliferation of a cell expressing RIP2 in the absence of the candidate compound, upon stimulation with IL-2 or Concanavalin A, alone or together with IL-1β.
c. contacting a cell which does not express RIP2 with the candidate compound and measuring cell proliferation, upon stimulation with IL2 or Concanavalin A, alone or together with IL-1β; and
d. comparing the cell proliferation in step (c) with the proliferation of a cell which does not express RIP2 in the absence of the candidate compound upon stimulation with IL-2 or Concanavalin A, alone or in combination with IL-1β;
wherein cell proliferation measured in step (a) is less than in step (b), and cell proliferation in step (c) is comparable to step (d) indicates that the compound is a RIP2 inhibitor.

38. The method of claim 37, wherein the contacting is conducted under conditions appropriate for entry of the candidate compounds into the cells.

39. A method of determining whether a compound is a RIP2 inhibitor comprising:
a. contacting a cell expressing RIP2 with a candidate compound and measuring the amount of the cell's IL-2 production upon T cell receptor stimulation;
b. comparing the amount of IL-2 measured in step (a) with an amount of the cell's IL-2 production in the absence of the candidate compound upon T cell receptor stimulation;
c. contacting a cell which does not express RIP2 with the candidate compound and measuring the cell's IL-2 production upon T cell receptor stimulation; and
d. comparing the amount of IL-2 measured in step (c) with an amount of IL-2 produced by a cell that does not express RIP2 in the absence of the candidate compound upon T cell receptor stimulation;
wherein an amount measured in step (a) is less than step (b), and an amount measured in step (c) is comparable to the amount measured in step (d) indicates that the compound is a RIP2 inhibitor.

40. The method of claim 39, wherein the contacting is conducted under conditions appropriate for entry of the candidate compounds into the cells.

41. A method of determining whether a compound is a RIP2 inhibitor comprising:
a. contacting cells expressing RIP2 with a candidate compound and measuring proliferation of the cells upon T cell receptor stimulation;
b. comparing the amount of proliferation measured in step (a) with the proliferation of cells expressing RIP2 in the absence of the candidate compound upon T cell receptor stimulation;
c. contacting cells which do not express RIP2 with the candidate compound and measuring the proliferation of the cells upon T cell receptor stimulation;
d. comparing the amount of proliferation measured in step (c) with the proliferation of cells which do not express RIP2 in the absence of the candidate compound upon T cell receptor stimulation;
wherein an amount measured in step (a) is less than step (b), and an amount measured in step (c) is comparable to the amount measured in step (d) indicates that the compound is a RIP2 inhibitor.

42. The method of claim 39 or 41, wherein the cell is a T cell.

43. An isolated cell which does not express RIP2.

44. The isolated cell of claim 43, wherein the cell is a mammalian cell.

45. The isolated cell of claim 43, wherein the cell is a fibroblast, T cell or macrophage.

46. The isolated cell of claim 43, wherein the cell is a mouse or human cell.

47. An isolated cell which does not normally express RIP2, which comprises an exogeneous nucleic acid encoding RIP2.

48. The isolated cell of claim 47, wherein the nucleic acid is contained in an expression vector.

49. The isolated cell of claim 43, obtained from a RIP2 deficient transgenic nonhuman animal.

50. The isolated cell of claim 49, wherein the transgenic non-human animal is a mouse.

51. The isolated cell of claim 49, wherein the transgenic non-human animal is a homozygous RIP2 deficient transgenic non-human animal.

52. The method of claim 33, wherein the NF-κB substrate is p38, IαBα, ERK or JNK.
